(19) United States
(12) Patent Application Publication
(10) Pub. No.: US 2011/0190677 A1
(43) Pub. Date: Aug. 4, 2011

(54) Garment

(75) Inventors: Nils STELTER, Frasdorf (DE); Petra Rieble-Hübner, Grobkarolinenfeld (DE)

(73) Assignee: AMOENA MEDIZIN-ORTHOPADIE-TECHNIK GMBH, Raubling (DE)

(21) Appl. No.: 13/009,339

(22) Filed: Jan. 19, 2011

(30) Foreign Application Priority Data
Feb. 1, 2010 (DE) 10 2010 006 459.9

(51) Int. Cl. A61F 13/10

(52) U.S. Cl. 602/42

(57) ABSTRACT

The present invention relates to a garment consisting of or comprising a least one tubular garment part to be stripped over an arm, with at least one curved joint region adapted to the shape of the arm joint, wherein this curved joint region is formed by stitches added on one side.

The present invention relates to a garment consisting of or comprising at least one tubular garment part to be slipped over an arm.

Such garments are used for example as compression arm sleeves in connection with surgery aftercare, such as in the aftercare of surgical treatments of breast cancer. In such procedures it may furthermore indicated, for example, to not only remove the breast affected by breast cancer, but also one or more lymph nodes in the upper arm of the patient on the respective side of the body.

In connection with these procedures special care is then taken during the surgery aftercare to provide for a good rehabilitation of the patient by using a compression, arm sleeve and to avoid possible complications such as edema formation and/or thromboses.

The compression arm sleeves known so far, however, have the disadvantage that on bending the arm a constriction occurs in particular in the crook of the arm, as in the bent condition the compression arm sleeve presses into the arm on the inside of the elbow especially due to the formation of creases.

Furthermore, the wear comfort of the known compression arm sleeves needs to be improved, since the material used so far have poor breathing properties, which is perceived as unpleasant. In addition, this can lead to the fact that the sweating tendency is increased. In case of doubt, this can furthermore lead to the fact that rashes and itching or an impairment of the healing process may occur.

Therefore, it is the object of the present invention to develop a garment as mentioned above in an advantageous way, in particular to the effect that the wear comfort can be increased, and furthermore to the effect that a pinch-off in the crook of the arm can safely be avoided.

In accordance with the invention, this object is solved by a garment with the features of claim 1. Accordingly, it is provided that a garment consists of at least one tubular garment part to be stripped over an arm or comprises at least one tubular garment part to be stripped over an arm. The garment part includes at least one curved joint region adapted or adaptable to the shape of an arm joint.

It thereby advantageously becomes possible to avoid a constriction in the joint region of the arm when bending or flexing the respective joint. The arm joint for example can also be hand joint, a shoulder joint or an elbow joint. Avoiding a constriction in the joint region in particular provides the further advantage that the circulation in the arm is distinctly improved. In particular, the term circulation is meant to also include the lymph flow beside the blood flow.

It can be provided that at least one joint region is adapted to the shape of an elbow joint and/or that distal of the joint region distal region, in particular a forearm region, and/or proximally a proximal, region, in particular an upper arm region, is located. Particularly advantageously it can thus be provided that to avoid slipping together of the sleeve in the elbow region, the garment is manufactured in a curved shape following the anatomy of the human arm. In this connection it is conceivable to produce the curvature by a special knitting technique in the region of the elbow, for example analogous to the formation of a heel part of a stocking or a sock. At the same time, this provides a fixation of the garment on the arm, as due to the given curvature of the bent joint region the positioning of the garment on the arm is predefined.

In addition, it is possible that the at least one garment part is formed at least partly elastic, in particular at least partly consists or is formed of elastic fibers and/or is manufactured by a circular knitting method, in particular without lateral seams. The manufacture by the circular knitting method without lateral seams in particular provides the advantage that the wear comfort can be increased. The absence of lateral seams provides for a uniform fit of the garment on the arm and the formation of bruises on the arm is avoided. In addition, it be provided that the garment at least partly consists of Lycra or Lycra fibers. By using Lycra it becomes possible for example to predetermine the elasticity of the garment in a certain region, i.e. to adjust a predefined pretension of a region of the garment.

Advantageously, it is provided that the elastic fibers and/or the Lycra fibers have a different pretension in certain regions. In this connection it is in particular conceivable that there is a first region which has a higher pretension than a second region of the garment part to be stripped over the arm.

The garment can consist of a continuous, plain knit ground fabric. In the region of the elbow, stitches preferably are additionally added on one side at a distance of 3 rows. These additional half rows marked in color are added to the ground fabric, whose length is variable, for example on a length of about 14 cm, and lead to the desired anatomical shape, i.e. shape adapted to the respective anatomy.

In the region of the crook, the knit fabric has the same density as in the regions of the upper arm and forearm, respectively. The elbow region is reinforced by the half rows and also longitudinally and transversely elastic. The Lycra preferably employed in the entire garment supports the fit and reduces the formation of creases. Due to the longitudinal and transverse elasticity, the garment possesses excellent wear properties and perfectly adapts to the respective anatomy.

Furthermore, it can be provided that the pretension is decreasing from the distal region of the garment part towards the proximal region, so that preferably a conical course of the supporting force is obtained and/or wherein furthermore preferably due to the pretension the supporting force in the distal region preferably is about 15-30 mmHg, advantageously about 20-27 mmHg, particularly preferably about 22-25 mmHg. Due to a conical course of the supporting force with a higher pretension in the distal region, which decreases towards the proximal region, the advantage is obtained that in patients in the condition after a lymph node resection the operation area in the region of the arm pit is compressed less and thereby spared due to the reduced pretension. At the same time, an edema for example can be prevented by such support as a result of the higher pretension in the distal region of the forearm, i.e. in the region close to the hand.

It is particularly preferred when the garment is a medical arm support sleeve or compression arm sleeve, which preferably extends from the hand joint to under the arm pit and is formed to be stripped over the arm.

Furthermore, it is conceivable that the garment, is at least partly made of latent heat storage material for temperature regulation and/or includes at least one latent heat accumulator for temperature regulation.

This involves the advantage that the wear comfort of the garment can distinctly be increased. Due to the latent heat accumulator it becomes possible to avoid or delay overheating, but also cooling off. The latent heat accumulator preferably can be a PCM material (phase-change material), i.e. a material in which a phase change takes place in the desired temperature range. Such phase change, for example from solid toy liquid or vice versa, has the advantage that with a constant material temperature in the temperature range of the phase change the material can absorb or release thermal energy.

The heat radiated by the body can be stored, without the temperature of the garment being increased or the heat being encapsulated, so that overheating can occur. The heat from the surroundings also can first be stored temporarily by the one or more latent heat accumulators. Cooling off for example is delayed or prevented in that in this case the temporarily stored heat is returned to the body.

Due to the one or more latent heat accumulators, the garment thus advantageously can perform the function of a heat buffer. Thus, reduced sweating occurs, since the temperature substantially can be kept constant, so that the body's own temperature regulating mechanisms comprising the formation of sweat need not intervene. Furthermore, the advantage is obtained that a uniform distribution of heat and an active temperature exchange are possible. Furthermore, it is advantageous that in the case of a medical use of the garment, for example in surgery aftercare, the healing process can be supported and accelerated, as by providing for a constant temperature the occurrence of itching or a possible inflammation can be prevented by the reduced stimulation of sweating.

Furthermore, it can be provided that the latent heat accumulator and/or the latent heat storage material at least partly comprises paraffin. Paraffin has the advantage that it has very good latent heat storage properties at body temperature. In addition, this substance is harmless in terms of handling and when packed properly can easily be used for medical applications and in particular can be sterilized and/or disinfected. The term paraffin also comprises paraffin-like substances such as waxes or wax-containing substances, which substantially have latent heat storage properties comparable to paraffin.

In addition, it is possible that the latent heat accumulator and/or the latent heat storage material at least partly includes a salt solution. This substance also is harmless in terms of handling and when packed properly also can easily be used for medical applications and in particular can be sterilized and/or disinfected. The salt solution for example can be a sodium chloride solution.

It is furthermore conceivable that the latent heat accumulator and/or the latent heat storage material are arranged in one or more capsules, in particular in microcapsules. The microcapsules preferably can be microscopically small, so that a high packing density can be obtained.

It is possible that the latent heat accumulator and/or the latent, heat storage material is incorporated into a yarn and/or a non-woven layer or fleece. It is particularly advantageous when the microcapsules in which the latent heat storage material such as paraffin is encapsulated are incorporated into the yarn or the non-woven layer, so that the yarn or non-woven layer can, be processed as usual. The yarn can be used for manufacturing a fabric from which the parts of the garment are made. It is possible, for example, to weave such a fabric exclusively by means of the yarn or to manufacture such fabric by means of the yarn and by additionally using further materials.

Furthermore, it can be provided that the latent heat accumulator and/or the latent heat storage material it present as coating and/or layer of the garment and/or is applied onto the garment. This coating or layer advantageously can be formed by a multitude of microcapsules in which the latent heat storage material such as paraffin is encapsulated.

It is also possible to provide two kinds of latent heat accumulators and/or latent heat storage material. It can be provided, for example, that a first kind of a latent heat accumulator or latent heat storage material has a first phase transition temperature and a second kind of a latent heat accumulator or latent heat storage material has a second phase transition temperature which differs from the first phase transition temperature, in particular slightly, by about 2-5° C. The wear comfort thereby can distinctly be increased, because the temperature range in which the latent heat accumulators perform the function of a heat-buffer is increased.

In addition, it is conceivable that the inner and/or outer surfaces of the garment at least partly include a latent heat storage material and/or a latent heat accumulator for temperature regulation. The inner surfaces for example are the surfaces which directly lie on the body. Preferably, the surfaces can be formed by fabrics which consist of yarn into which microcapsules are incorporated, in which the latent heat storage material such as paraffin is encapsulated. The surface likewise can be formed by fabrics which consist, of non-woven layers in which corresponding microcapsules are incorporated. In this connection it is furthermore advantageously conceivable that the surfaces located outside in the wear condition, i.e. the outer surfaces, of the garment, are at least partly coated with microencapsulated latent heat storage material. It can also be provided that the entire inner surfaces of the garment consist of a fabric with yarn with latent heat storage material and/or consist of a non-Woven layer with latent heat storage material. It is also conceivable that the entire inner surfaces of the garment have a coating with latent heat storage material.

Preferably, it can furthermore be provided that at least the garment part consists of a yarn with microencapsulated latent heat storage material for more than 40%, advantageously for more than 45%, particularly preferably for more than 50%.

Further details and advantages of the invention now be explained in detail with reference to an embodiment illustrated in the drawing,

The only FIGURE shows a schematic representation of the garment of the invention in an advantageous embodiment.

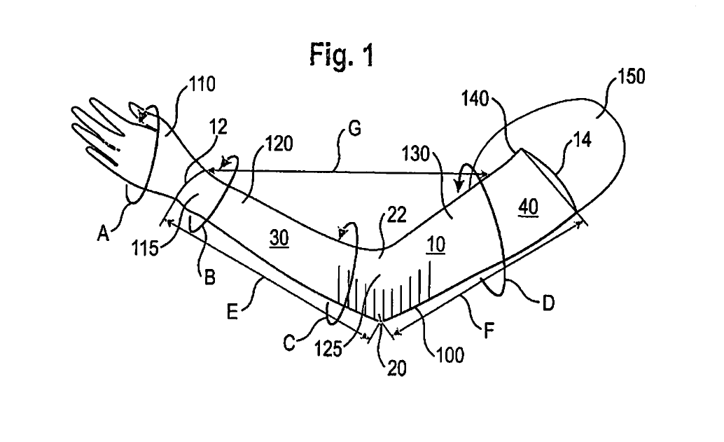

FIG. 1 shows a schematic representation of a medical compression arm sleeve 10 made by a circular knitting method, which has no lateral seams, i.e. is made seamless. The compression arm sleeve 10 is designed as a compression arm sleeve 10 for a left arm 100 and preferably furthermore available in a non-illustrated, in particular symmetrical design for the right arm. It is, however also conceivable that for the left arm and the right arm a single embodiment of a compression arm sleeve 10 is provided.

To put on the compression arm sleeve 10, the same is pulled over the hand 110 onto arm 100 and in the applied condition extends from the hand joint 115 to under the arm pit 140, wherein the lower cuff 12 lies flat directly above the hand joint 115 and the upper cuff 14 of the compression arm sleeve 10 reaches under the arm pit 140. Thus, the forearm 120 and the upper arm 130 are completely enclosed by the compression arm sleeve 10, whereas the hand 110 and the shoulder 150 are exposed.

The compression arm sleeve 10 has a distal region 50 or a forearm region 30, which has a length E measured from the elbow joint 125 to the lower cuff 12, and a proximal region 40 or an upper arm region 40, which has a length F as measured from the elbow joint 125 to the upper cuff 14.

In the region of the elbow 125 the compression arm sleeve 10 has a curved joint region 20 adapted to the shape of the elbow joint 125, which other than the remaining regions of the compression arm sleeve 10 is knitted in a particular way, a different way as compared to the remaining regions of the compression arm sleeve 10.

The compression arm sleeve 10 consists of a continuous, plain knit ground fabric. In the joint region 20 stitches are additionally added on one side at a distance of 3 rows. These additional half rows marked in color are added to the ground fabric, whose length is variable, on a length of about 14 cm, and lead to the desired anatomical shape of the compression arm sleeve 10. In the region of the crook 22, the knit fabric has the same density as in the regions of the upper arm and forearm 30, 40, respectively. The elbow region 20 is reinforced by the half rows and also longitudinally and transversely elastic.

Due to this three-dimensional shaping of the compression arm sleeve 10 in the joint region 20 it becomes possible that a reduction of the formation of creases in the region of the crook of the arm occurs, which involves an increased wear comfort and at the same time prevents a constriction of the arm 100. The Lycra employed in the entire compression arm sleeve 10 supports the fit and reduces the formation of creases. Due to the longitudinal and transverse elasticity, the compression arm sleeve 10 possesses excellent wear end perfectly adapts to the respective anatomy.

The curvature in the unloaded condition is determined by the lengths E, F and G, so that in the embodiment shown here an angle of curvature of about 100° is obtained.

In the illustrated embodiment, the compression arm sleeve 10 consists of a yarn with microencapsulated latent-heat storage material for more than 50%. This latent heat storage material is paraffin which is directly incorporated into the yarn in microcapsules. Furthermore, Lycra fibers with a defined pretension are processed, in order to produce a conical course of the supporting force, wherein the course of the supporting force is decreasing from the hand joint 115 the upper arm 130. The supporting force of the compression arm sleeve 10 in the region of the hand joint 115 is about 22-25 mmHg.

Due to the elasticity of the compression arm sleeve 10 it is easily possible to move the arm 100, for example during physiotherapeutic exercises. The possible movements of the arm 100 are not completely indicated by the arrows A, B, C and D. Of course, the elasticity of the compression arm sleeve 10 also allows further movements.

1. A garment comprising at least one tubular garment part to be stripped over an arm, the tubular garment part including at least one curved joint region adapted to the shape of the arm joint, wherein
the curved joint region is formed by stitches added on one side.

2. The garment according to claim 1, wherein the stitches added on one side have a distance of three rows.

3. The garment according to claim 1, wherein the stitches added on one side have a variable length.

4. The garment according to claim 1, wherein the at least one joint region is adapted to the shape of an elbow joint.

5. The garment according to claim 1, wherein the at least one garment part is at least partly elastic.

6. The garment according to claim 5, wherein the garment is at least partly made of Lycra or Lycra fibers.

7. The garment according to claim 15, wherein the elastic fibers have a different pretension in certain regions.

8. The garment according to claim 7, wherein the pretension is decreasing from a distal region of the garment part which is adapted to cover at least a portion of a forearm region towards a proximal region, which is adapted to cover at least a portion of an upper arm region so that a conical course of supporting force is obtained.

9. The garment according to claim 1, wherein the garment is a medical arm support sleeve or compression arm sleeve, which extends from a hand joint to under an arm pit and is formed to be stripped over the arm.

10. The garment according to claim 1, wherein the garment is at least partly made of latent heat storage material for temperature regulation and/or includes at least one latent heat accumulator for temperature regulation.

11. The garment according to claim 10, wherein the latent heat accumulator and/or the latent heat storage material at least partly comprises paraffin and/or that the latent heat accumulator and/or the latent heat storage material at least partly includes a salt solution.

12. The garment according to claim 10, wherein the latent heat accumulator and/or the latent heat storage material are arranged in one or more capsules.

13. The garment according to claim 4, wherein the joint region includes an forearm region.

14. The garment according to claim 4, wherein the joint region includes an upper arm region.

15. The garment according to claim 5, wherein the at least one garment part includes elastic fibers.

16. The garment according to claim 1, wherein the at least one garment part is manufactured by circular knitting without lateral seams.

17. The garment according to claim 8, wherein the supporting force due to the pretension in the distal region is about 15-30 mmHg.

18. The garment according to claim 17, wherein the supporting force due to the pretension in the distal region is about 20-27 mmHg.

19. The garment according to claim 18, wherein the supporting force due to the pretension in the distal region is about 22-25 mmHg.

20. The garment according to claim 12, wherein the capsules are microcapsules.

21. The garment according to claim 12, wherein the latent heat accumulator and/or the latent heat storage material is incorporated into a yarn and/or a non-woven layer or fleece.

22. The garment according to claim 20, wherein at least the garment part consists of a yarn with microencapsulated latent heat storage material for more than 40%.

23. The garment according to claim 22, wherein at least the garment part consists of a yarn with microencapsulated latent heat storage material for more than 45%.

24. The garment according to claim 23, wherein at least the garment part consists of a yarn with microencapsulated latent heat storage material for more than 50%.
